(19) United States
(12) Patent Application Publication
(10) Pub. No.: US 2013/0316180 A1
(43) Pub. Date: Nov. 28, 2013

(54) Biocompatible Packaging Suitable for Long-term Implantation and Method of Formation

(71) Applicant: Case Western Reserve University, Euclid, OH (US)

(72) Inventors: Wen H. Ko, Cleveland Heights, OH (US); Shem Lachman, Old Westbury, NY (US); Christian A. Zorman, Euclid, OH (US); Leping Bu, Wuhan (CN)

(73) Assignee: Case Western Reserve University, Euclid, OH (US)

(21) Appl. No.: 13/888,731

(22) Filed: May 7, 2013

Related U.S. Application Data
(60) Provisional application No. 61/643,647, filed on May 7, 2012.

(51) Int. Cl. A61L 27/34

(52) U.S. Cl. CPC A61L 27/34
USPC 428/414; 428/411.1; 428/447; 428/413; 427/2.11

(57) ABSTRACT

A method for forming an electrical-conductor-free vapor barrier suitable for protecting long-term implanted electronic systems is disclosed. The method comprises forming a nascent layer of a partially cured layer and repeatedly compressing the layer via a roller-based process. Once the layer has been suitably compressed, the layer is fully cured. In some embodiments, a multi-layer protective layer is formed by repeating the roller-based formation process for each of a plurality of layers. In some embodiments, a multi-layer protective layer comprising layers of different materials is formed.

### CROSS REFERENCE TO RELATED APPLICATIONS

moon This case claims priority of U.S. Provisional Patent Application U.S. 61/643,647, which was filed on May 7, 2012 (Attorney Docket: 747-007US), and which is incorporated herein by reference.

### STATEMENT REGARDING FEDERALLY SPONSORED RESEARCH

This invention was made with Government support under Contract Number 1R21EB014442-01 awarded by the National Institute of Health. The Government has certain rights in the invention.

### FIELD OF THE INVENTION

The present invention relates to packaging suitable for long-term protection of electronic circuits and devices in general, and, more particularly, to packaging for implantable biomedical sensors and systems.

### BACKGROUND OF THE INVENTION

Although developments in Micro-Electro-Mechanical-Systems (MEMS) technology have enabled physical, chemical, and electrical sensor systems of staggering complexity to be formed on chips having very small footprints, the challenges inherent in packaging such systems has impeded their adoption in many application areas, including aerospace, medicine, and industrial controls.

In the medical field, for example, implantation of a sensor and/or electronic system into living tissue subjects the system to a biochemical environment that can lead to rapid failure of the MEMS devices. In addition, for long-term implantation, exposure of the living tissue to the foreign matter of the MEMS device can induce generation of defense mechanisms, such as inflammation, fibro-collagenous capsule build-up around the sensor chip, that inhibit or negate the operation of the sensors and electronic systems. As a result, the packaging used to protect implantable sensor and systems has a critical role in determining the viability of such systems.

The package of an implantable system has two functions: first, to protect the implanted device from damage due to the host; and second, to protect the tissue and host environment from harm or other undesirable effects due to the implanted device. Packaging issues include: physical considerations, such as surface softness and specific gravity, transmission of heat to/from the sensor, mechanical strength, etc.; chemical considerations, such as water, water vapor, ionic, and ionic vapor permeation; and biological considerations, such as toxicity of materials in the implanted device, and irritation due to packaging features.

As a result, it is desirable that an implantable sensor package includes an outer-layer material having a mechanical stiffness similar to the tissue surrounding the package, a shape and surface that it does not produce large stress and strain on the interface tissues, and that avoids significant transmission of heat from hot spots on the sensor chip to the surrounding tissue. In addition, an implantable sensor package must be free of toxic materials that could leach out to the surrounding tissue and result in inflammation and or unacceptable pathological reactions around the implant site. Further, the package must be sterile, containing no biological elements (e.g., virus, proteins, etc.).

Unfortunately, to date, no single homogeneous material has been identified that satisfies all of these requirements for implantable MEMS systems. Typically, prior-art sensors and systems are packaged with a multi-part exterior including (1) a substantially hermetic seal provided by a metallic case that mitigates vapor penetration through the package and (2) a silicone-like material outer coating that affords better tissue compatibility.

Examples of a prior-art packaging approach that includes a metal seal are disclosed by B. Mech, et al., in U.S. Patent Publication No. 2006/0173497, wherein an inner inorganic insulating layer is over coated with a bio-compatible protective layer of metal.

While the outer layer of metal mitigates dissolution of the polymer inner layer, it makes wireless communication to and from the enclosed system difficult, if not impossible. Further, the metal layer adds to the volume and weight of the system. Thus, such prior-art encapsulated sensors are not well suited for long-term implantation or for implantation in or on an internal organ.

Alternative prior-art packaging approaches include multi-layer coatings wherein an inorganic layer is sandwiched between biocompatible parylene layers, such as is disclosed by A. Hogg, et al., in U.S. Pat. No. 8,313,819. Each of the layers is deposited via vapor deposition so as to form a thin layer of material. The barriers between different layers are relied upon to dominate diffusion behavior of contaminants through the multi-layer structure, while the inorganic layer is relied upon to enhance the effectiveness of the barrier due to its highly dense nature.

Unfortunately, many inorganic materials are unacceptable in some applications. Further, vapor deposited layers can have high pinhole density in their as-deposited form. In addition, particulates trapped in a vapor deposited thin layer can rupture adjacent layers in the multi-layer structure, thereby creating pathways for contaminants through the barrier. Still further, the bonding strength between inorganic layers and adjacent parylene layers is often weak. As a result, delamination can occur when the layers are subjected to relatively low bending stress. Such issues can degrade the integrity of the barrier provided by such multi-layer coatings and lead to a shortened device lifetime. Thus far, these drawbacks of prior-art encapsulated systems is typically have limited their implantation duration to a few days or weeks.

There exists a need, therefore, for a biocompatible packaging technology suitable for implantable, wireless MEMS sensors systems, for chronic implants.

### SUMMARY OF THE INVENTION

The present invention enables a wireless electronic system without some of the costs and disadvantages of the prior art. Embodiments of the present invention are particularly well suited for use in medical, aerospace, semiconductor device, and industrial applications.

An illustrative embodiment of the present invention comprises an electrical-conductor-free protective barrier for an implantable electronic system, wherein the barrier includes a pressure-densified layer of organic coating material, such as silicone, that is formed over a circuit disposed on a printed-circuit board. The pressure-densified layer is formed as a thin layer on the circuit using a roller-deposition method. The layer is densified on each surface of the substrate underlying the layer by repeatedly compressing its thickness via a roller while pressure is applied between the roller and each substrate surface. After it has been cured, the coating process is repeated to form additional layers of densified films, thereby creating a multilayer barrier.

It is an aspect of the present invention that layer densification and/or multilayer formation can significantly improve adhesion of the protective layer to the underlying surfaces, reduce or eliminate voids between the barrier and regions of the system substrate, thereby mitigating the potential for water formation in these unbounded crevices, mitigate the effects of particulate contamination, and generally improve layer reliability and performance.

In some embodiments, the pressure-densified layer is a layer of epoxy suitable for use in integrated circuit packaging.

In some embodiments, a multi-layer protective layer stack is formed on an electronic system, wherein each layer is a layer of organic material, such as parylene C, epoxy, silicone, and the like. In some embodiments, a multi-layer protective layer stack is includes two or more layers of parylene C, wherein a pressure-densified layer of organic material is disposed between two of these parylene C layers.

An embodiment of the present invention comprises a method for forming a protective layer, the method comprising: forming a first layer of a first material on a substrate that defines a first plane, the first layer having a first surface; applying a first compressive force to the first layer, the first compressive force having a force component that is aligned with a first direction that is substantially normal to the first plane; and curing the first material.

### BRIEF DESCRIPTION OF THE DRAWINGS

FIG. 1 depicts a schematic drawing of a cross-sectional view of a sensor system in accordance with an illustrative embodiment of the present invention.

FIG. 2 depicts a schematic drawing of a cross-sectional view of a protective layer in accordance with the illustrative embodiment of the present invention.

FIG. 3 depicts operations of a method suitable for the formation of a protective layer in accordance with the illustrative embodiment of the present invention.

FIGS. 4A-B depict schematic drawings of a cross-sectional view of layer 202-i at different points in its fabrication in accordance with the illustrative embodiment of the present invention.

FIG. 5 depicts a schematic drawing of a cross-sectional view of a protective layer in accordance with a first alternative embodiment of the present invention.

FIG. 6 depicts a schematic drawing of a cross-sectional view of a protective layer in accordance with a second alternative embodiment of the present invention.

FIG. 7 depicts a schematic drawing of a cross-sectional view of a protective layer in accordance with a third alternative embodiment of the present invention.

FIG. 8 depicts a schematic drawing of a cross-sectional view of a protective layer in accordance with a fourth alternative embodiment of the present invention.

### DETAILED DESCRIPTION

FIG. 1 depicts a schematic drawing of a cross-sectional view of a sensor system in accordance with an illustrative embodiment of the present invention. System 100 comprises substrate 102, microsystem 104, and protective layer 106. System 100 is a medical sensor suitable for long-term implantation into a human body. “Long-term implantation,” in the context of this Specification, means from several (i.e., at least two) months to several years. In some embodiments, system 100 is an electronic system other than a medical sensor.

Substrate 102 is a conventional silicon substrate comprising traces suitable for conveying electrical signals and/or power to, from, and within microsystem 104. Substrate 102 defines plane 110, which is substantially parallel to the major surfaces of the substrate. In some embodiments, substrate 102 is a substrate, other than a silicon substrate, that is suitable for use in MEMS fabrication, nanotechnology, planar processing, and the like. Substrates suitable for use with the present invention include, without limitation, printed circuit boards, semiconductor substrates (including germanium, silicon carbide, III-V semiconductor, and II-VI semiconductor substrates), ceramic substrates, glass substrates, alumina substrates, and the like. In some embodiments, substrate 102 comprises electrical traces suitable for conveying high-frequency electrical signals, such as transmission lines. In some embodiments, substrate 102 does not include electrical traces. In some embodiments, system 100 includes a sensor that is other than a MEMS-based sensor.

Microsystem 104 is a MEMS-based electronic system comprising one or more MEMS transducers, such as physical and/or chemical sensors, or actuators, and associated electronic circuitry. In some embodiments, microsystem 104 comprises circuitry for purposes such as controlling one or more transducers, amplifying and/or conditioning output signals from the one or more sensors, and the like. In some embodiments, microsystem 104 includes one or more wireless transceivers for transmitting and/or receive wireless information to/from module 104.

In some embodiments, microsystem 104 is formed on a substrate other than substrate 102 and this different substrate is attached to substrate 102 via a conventional hybrid bonding technique, such as solder-bump bonding, epoxy attachment, wafer bonding (e.g., thermo-anodic, fusion, etc.), and the like.

Protective layer 106 is a pressure-densified layer of silicone or silicone compound having a thickness on substrate surface 108 that is within the range of approximately 30 microns to approximately 100 microns, and preferably approximately 50 microns. Typically, in order to fully protect substrate 102, protective layer 106 completely surrounds the substrate (i.e., is disposed on all 6 sides of the substrate). For the purposes of this Specification, including the appended claims, a “pressure-densified layer” is defined as a layer of material whose thickness has been mechanically compressed from its nascent, as-deposited thickness by applying compressive force directed through the thickness of the nascent layer via a mechanical tool, such as a roller, wire-wound rod, etc.

In some embodiments, protective layer 106 comprises a material other than a silicone, wherein the material is suitable for mitigating exposure of microsystem 104 to undesirable environmental conditions. Materials suitable for use in protective layer 106 include, without limitation, silicone compounds (e.g., PDMS, etc.), medical-grade epoxy, organic polymer encapsulants, and the like. In some embodiments, protective layer 106 is a multi-layer coating comprising one or more layers of a plurality of suitable coating materials.

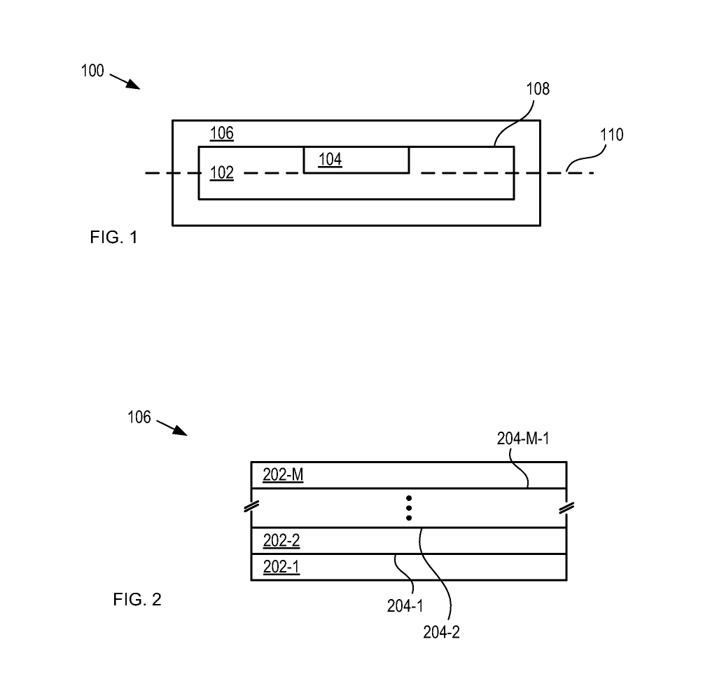

FIG. 2 depicts a schematic drawing of a cross-sectional view of a portion of a protective layer in accordance with the illustrative embodiment of the present invention. Protective layer 106 comprises layers 202-1 through 202-M, where M is any practical integer number of sub-layers.

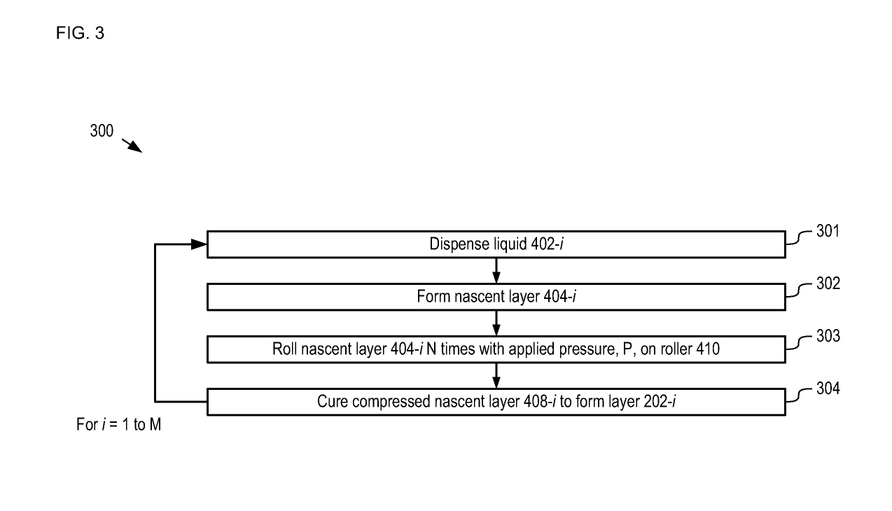

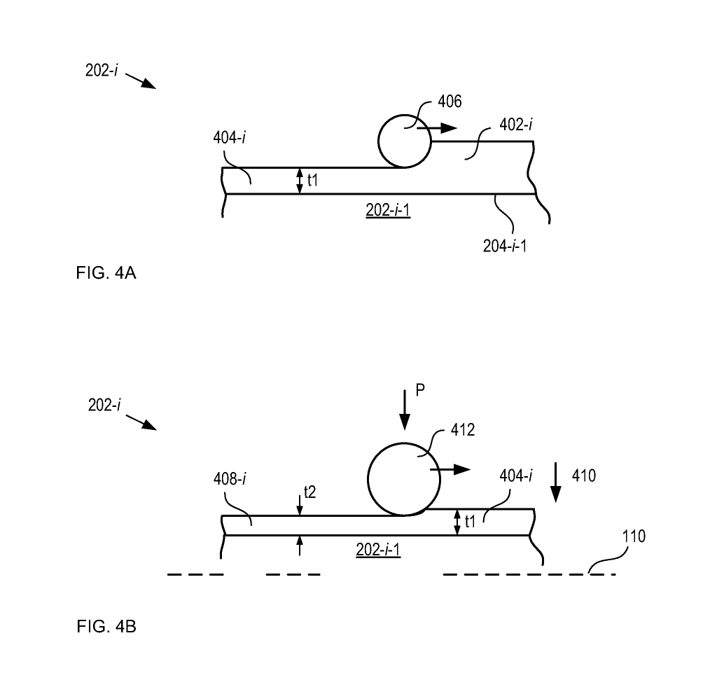

FIG. 3 depicts operations of a method suitable for the formation of a protective layer in accordance with the illustrative embodiment of the present invention. Method 300 begins with operation 301, wherein material 402-i is disposed on surface 204-i-1. FIG. 3 is described with continuing reference to FIGS. 1-2 and with reference to FIGS. 4A-B.

FIGS. 4A-B depict schematic drawings of a cross-sectional view of layer 202-i at different points in its fabrication in accordance with the illustrative embodiment of the present invention.

At operation 302, nascent layer 404-i is formed by spreading material 402-i on surface 204-i-1 to first desired thickness, t1, via roller 406. First thickness, t1, is within the range of approximately 3 microns to approximately 20 microns, and typically about 10 microns. Note that for the formation of nascent layer 404-1, surface 204-i-1 is surface 108 of substrate 102. In some embodiments, nascent layer 404-i is formed via another conventional deposition method; such as spin coating, doctor blading, silk screening, vapor deposition, and the like. It should be noted that the method used to form nascent layer 404-i is often based on the material chosen for use in layer 202-i. For example, the preferred deposition method for parylene C is vapor deposition.

FIG. 4A depicts nascent layer 404-i during operation 302.

At operation 303, nascent layer 404-i is pressure densified to form compressed nascent layer 408-i, which constitutes a pressure-densified layer, as defined above.

FIG. 4B depicts nascent layer 404-i during operation 303.

In order to pressure densify nascent layer 404-i, the layer is rolled via roller 412 with pressure, P, applied to the layer such that a force component is generated along direction 410 (i.e., through the thickness of the layer toward substrate 102). In some embodiments, the applied pressure and the viscosity of liquid 402-i collectively determine the thickness of nascent layer 404-i after operation 303. In some embodiments, roller 412 is coated with additional liquid 402-i during operation 303. Although in some embodiments, a single rolling of nascent layer 404-i is can be used to densify the layer sufficiently, more typically nascent layer 404-i is rolled N times (wherein N is a number typically within the range of 1 to 500, and typically 200) by roller 412 while pressure P is applied to the roller. The compressive force directed through the thickness of nascent layer 404-i (i.e., along direction 410, as shown) results in a compression of nascent layer 404-i to second thickness t2. In some embodiments, t2 has a value within the range of approximately 10 microns to approximately 100 microns, and is typically within the range of approximately 30 microns to approximately 40 microns, and preferably approximately 50 microns. In some embodiments, roller 412 remains stationary while the substrate and nascent layer are moved relative to the roller. In some embodiments, pressure P is applied to nascent layer 404-i via roller pairs, calendars, or the like.

It is an aspect of the present invention that by rolling nascent layer 404-i with a roller while pressure is applied between the roller its underlying substrate, adhesion of nascent layer 404-i to the substrate, as well as any intervening structure/components, is improved.

It is a further aspect of the present invention, that mechanical force applied to each nascent layer 404-i reduces the deleterious effects of structural defects, such as small air bubbles, particulate, and the like, in the layer by either crushing them, fully encapsulating them with layer material, or driving them from the surface of nascent layer 404-i, thereby improving the overall integrity of the seal formed by the layer.

It should be noted that the performance of some prior-art encapsulation layers has been compromised by the presence of pinholes in the layer. Pinholes provide access for vapor and contaminants through the layer—either from within the electronic package to the surrounding tissue or from the surrounding environment into the electronic package. Although pressure densification has been employed in the prior art to increase the density of a layer, such as active electrode films described in U.S. Patent Publication No. 2006/0143884, the porosity of such layers is intentionally unchanged by the pressure-densification process. In such layers, porosity plays an important role in increasing the effective surface area even as the thickness of the layer decreases. As a result, such pressure-densification processes are not suitable for use with embodiments of the present invention since they do not improve the integrity of the pressure-densified layer as a contaminant barrier.

It is believed that that the mechanical force applied to each nascent layer 404-i reduces the number and effect of pinholes in the layer as the density of the layer is increased, thereby increasing the quality of the layer as a contaminant barrier. Although there is no direct evidence of reduced pinhole density, barriers in accordance with the present invention have been experimentally shown to exhibit 10 to 100 times longer life time than typical prior-art barriers in saline tests.

At operation 304, compressed nascent layer 408-i is fully cured, in conventional fashion, to form layer 202-i.

Operations 301 through 304 are then repeated for each surface of substrate 102 and for each successive layer 202-i in protective layer 106.

It is an aspect of the present invention that a multi-layer stack of thin layers is preferable to multi-layer stacks comprising one or more thick layers. As a result, the thickness of each of layers 202-1 through 202-M is kept thin—preferably, having a thickness of less than or equal to about 30 microns, as discussed above and with respect to layer 202-i.

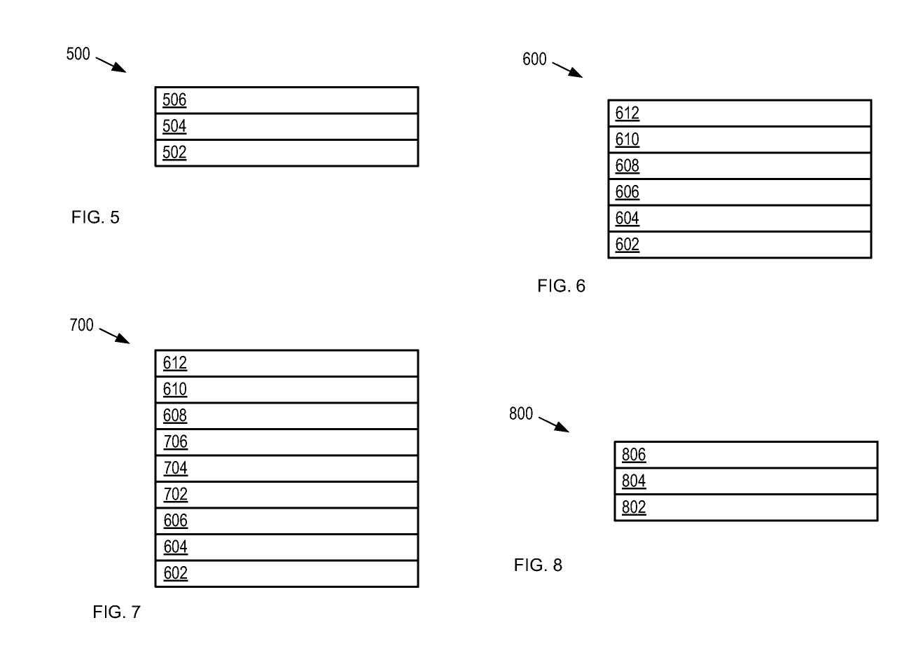

FIG. 5 depicts a schematic drawing of a cross-sectional view of a protective layer in accordance with a first alternative embodiment of the present invention. Protective layer 500 comprises layers 502, 504, and 506.

Each of layers 502, 504, and 506 is a thin film of silicone that is a pressure-densified layer formed via operations analogous to the operations of method 200. Typically, each of layers 502, 504, and 506 is rolled via an operation analogous to operation 203 N times (1≦N≦500) and fully cured prior to the formation of the layer disposed upon it.

In some embodiments, each of layers 502, 504, and 506 is a layer of medical-grade epoxy. In some embodiments, each of layers 502, 504, and 506 is a layer comprising a different organic material suitable for forming a protective layer in accordance with the present invention.

Typical thickness for each of layers 502, 504, and 506 is within the range of approximately 10 microns to approximately 100 microns, and is typically within the range of approximately 30 microns to approximately 40 microns, and preferably approximately 50 microns.

FIG. 6 depicts a schematic drawing of a cross-sectional view of a protective layer in accordance with a second alternative embodiment of the present invention. Protective layer 600 comprises layers 602, 604, 606, 608, 610, and 612.

Each of layers 602, 604, and 606 is a layer of medical-grade epoxy having a thickness within the range of approximately 50 microns to approximately 100 microns. In some embodiments, one or more of layers 602, 604, and 606 is a pressure-densified layer.

Each of layers 608, 610, and 612 is a pressure-densified layer of silicone formed via operations analogous to the operations of method 200. Typically, each of layers 608, 610, and 612 is rolled via a roller analogous to roller 410 N times (1≦N≦500) and fully cured prior to the formation of the layer disposed upon it.

Each of layers 608, 610, and 612 has a thickness within the range of approximately 30 microns to approximately 60 microns, and preferably 50 microns.

FIG. 7 depicts a schematic drawing of a cross-sectional view of a protective layer in accordance with a third alternative embodiment of the present invention. Protective layer 700 comprises layers 602, 604, 606, 702, 704, 706, 608, 610, and 612.

Each of layers 702, 704, and 706 is a pressure-densified layer of silicone formed via operations analogous to the operations of method 200. Typically, each of layers 702, 704, and 706 is rolled via a roller analogous to roller 410 N times (1≦N≦500) and fully cured prior to the formation of the layer disposed upon it. In some embodiments, each of layers 702, 704, and 706 is a pressure-densified layer of another organic material suitable for use with the present invention. In some embodiments, each of layers 702, 704, and 706 is a layer of parylene C.

Each of layers 702, 704, and 706 has a thickness within the range of approximately 5 microns to approximately 60 microns, and preferably 30 microns.

As a result, the total thickness of protective layer 700 is within the range of approximately 90 microns to approximately 540 microns, and preferably approximately 270 microns.

FIG. 8 depicts a schematic drawing of a cross-sectional view of a protective layer in accordance with a fourth alternative embodiment of the present invention. Protective layer 800 comprises layers 802, 804, and 806.

Layer 802 is a layer of parylene C having a thickness within the range of approximately 10 microns to approximately 60 microns, and preferably 50 microns.

Layer 804 is a layer of silicone having a thickness within the range of approximately 10 microns to approximately 60 microns, and preferably 30 microns. Layer 804 is a pressure-densified layer formed via operations analogous to the operations of method 200. In some embodiments, layer 804 comprises a material other than silicone, which is suitable for pressure-densification in accordance with the present invention.

Layer 806 is a layer of parylene C having a thickness within the range of approximately 10 microns to approximately 60 microns, and preferably 30 microns.

Typically, layers 802 and 804 are deposited via conventional vapor-phase deposition techniques and cured without their being subjected to pressure densification.

It should be noted that the layer structures described herein are exemplary only, and one skilled in the art will recognize that the order, thickness, and composition of the layers are matters of design choice. As a result, it will be clear to one skilled in the art, after reading this Specification, how to specify, make, and use alternative embodiments of the present invention wherein a protective layer, such as protective layer 106, includes any practical number of sub-layers having any practical order of materials, wherein one or more of the sub-layers is a pressure-densified layer.

It is to be understood that the disclosure teaches just one example of the illustrative embodiment and that many variations of the invention can easily be devised by those skilled in the art after reading this disclosure and that the scope of the present invention is to be determined by the following claims.

What is claimed is:

1. A method for forming an electrical-conductor-free protective layer on a substrate, the method comprising:
forming a first layer of a first material on a first surface of the substrate, the first layer having a second surface, wherein the first layer forms a barrier to at least one contaminant for the first surface;
applying a first compressive force that is directed through the thickness of the first layer, wherein the first compressive force is applied N times, where 1≦N≦500; and
curing the first material;
wherein the first layer forms a barrier to at least one contaminant for the first surface after the first material is cured.

2. The method of claim 1 wherein the first layer is provided such that the first material comprises a material selected from the group consisting of epoxy, silicone, and a silicone compound.

3. The method of claim 1 further comprising:
forming a second layer of a second material on the second surface, the second layer having a third surface;
applying a second compressive force that is directed through the thickness of the second layer, wherein the second compressive force is applied N times, where 1≦N≦500; and
curing the second material.

4. The method of claim 3 wherein each of the first material and second material comprises a material selected from the group consisting of epoxy, silicone, and a silicone compound.

5. The method of claim 3 further comprising providing the first material and the second material such that they are the same material.

6. The method of claim 1 wherein the first compressive force is applied by operations comprising:
positioning a first roller and the substrate such that the first roller is positioned against the second surface;
applying the first compressive force between the first roller and the substrate; and
inducing relative motion of the substrate and the first roller.

7. The method of claim 1 further comprising:
forming a second layer of a second material on the second surface, the second layer having a third surface;
applying a second compressive force that is directed through the thickness of the second layer, wherein the second compressive force is applied N times, where 1≦N≦500;
curing the second material;
forming a third layer of a third material on the third surface, the third layer having a fourth surface;
applying a third compressive force that is directed through the thickness of the third layer, wherein the third compressive force is applied N times, where 1≦N≦500; and
curing the third material.

8. The method of claim 7 further comprising:
forming a fourth layer of a fourth material disposed on the fourth surface, the fourth layer having a fifth surface;
applying a fourth compressive force that is directed through the thickness of the fourth layer, wherein the fourth compressive force is applied N times, where 1≦N≦500; and
curing the fourth material.

9. A method for forming an electrical-conductor-free protective layer, the method comprising:
forming a first layer on a substrate that defines a first plane, the first layer having a first surface, and the first layer comprising an epoxy;
forming a second layer on the first surface, the second layer having a second surface, and the second layer comprising the epoxy;
forming a third layer on the second surface, the third layer having a third surface, and the third layer comprising the epoxy;
forming a fourth layer on the third surface, the fourth layer having a fourth surface, and the fourth layer comprising parylene.

10. The method of claim 9 further comprising:
forming a fifth layer on the fourth surface, the fifth layer having a fifth surface, and the fifth layer comprising parylene; and
forming a sixth layer on the fifth surface, the sixth layer having a sixth surface, and the sixth layer comprising parylene.

11. The method of claim 10 further comprising:
forming a seventh layer on the sixth surface, the seventh layer having a seventh surface, and the seventh layer comprising silicone;
applying a fourth compressive force that is directed through the thickness of the seventh layer;
curing the seventh layer;
forming an eighth layer on the seventh surface, the eighth layer having a eighth surface, and the eighth layer comprising silicone;
applying a fifth compressive force that is directed through the thickness of the eighth layer;
curing the eighth layer;
forming a ninth layer on the eighth surface, the ninth layer having a ninth surface, and the ninth layer comprising silicone;
applying a sixth compressive force that is directed through the thickness of the ninth layer; and
curing the ninth layer.

12. A protective layer that is substantially electrical-conductor-free, the protective layer comprising:
a first pressure-densified layer comprising a first material, the first pressure-densified layer having a first surface;
a second pressure-densified layer comprising the first material, the second pressure-densified layer disposed on the first surface; and
a third pressure-densified layer comprising the first material, the third pressure-densified layer disposed on the second surface.

13. The protective layer of claim 12 wherein the first material comprises a material selected from the group consisting of silicone and epoxy.

14. The protective layer of claim 12 further comprising:
a fourth pressure-densified layer comprising a second material, the fourth pressure-densified layer having a fourth surface;
a fifth pressure-densified layer comprising the second material, the fifth pressure-densified layer disposed on the fifth surface; and
a sixth pressure-densified layer comprising the second material, the sixth pressure-densified layer disposed on the fifth surface.

15. The protective layer of claim 14 wherein the first material comprises epoxy and the second material comprises silicone.

16. The protective layer of claim 12 further comprising:
a first layer comprising parylene, the first layer being disposed between the first pressure-densified layer and a substrate.

17. The protective layer of claim 16 further comprising:
a second layer comprising the parylene, the second layer being disposed between the first pressure-densified layer and the second pressure-densified layer.

18. The protective layer of claim 12 further comprising:
a first layer comprising epoxy, the first layer being disposed between the first pressure-densified layer and a substrate.

19. The protective layer of claim 18 further comprising:
a fourth pressure-densified layer comprising a second material, the fourth pressure-densified layer having a fourth surface;
a fifth pressure-densified layer comprising the second material, the fifth pressure-densified layer disposed on the fifth surface; and
a sixth pressure-densified layer comprising the second material, the sixth pressure-densified layer disposed on the fifth surface.

20. The protective layer of claim 19 wherein the first material comprises a material selected from the group consisting of silicone and epoxy and the second material comprises a material selected from the group consisting of silicone and epoxy.
